(19) United States
(12) Patent Application Publication
(10) Pub. No.: US 2022/0079538 A1
(43) Pub. Date: Mar. 17, 2022

(54) X-RAY DYNAMIC IMAGE DISPLAY APPARATUS, STORAGE MEDIUM, X-RAY DYNAMIC IMAGE DISPLAY METHOD, AND X-RAY DYNAMIC IMAGE DISPLAY SYSTEM

(71) Applicant: Konica Minolta, Inc., Tokyo (JP)

(72) Inventor: Akinori TSUNOMORI, Tokyo (JP)

(21) Appl. No.: 17/475,745

(22) Filed: Sep. 15, 2021

(30) Foreign Application Priority Data
Sep. 15, 2020 (JP) 2020-154220

(51) Int. Cl. A61B 6/00; H04N 5/262

(52) U.S. Cl. CPC A61B 6/463; A61B 6/5247; H04N 5/262

(57) ABSTRACT

An X-ray-dynamic-image display apparatus includes a first hardware processor that: obtains X-ray-dynamic-image related information on an X-ray dynamic image obtained through X-ray dynamic imaging and camera-moving-image related information on a camera moving image obtained through camera imaging; and synchronously displays the X-ray-dynamic-image related information and the camera-moving-image related information.

### CROSS-REFERENCE TO RELATED APPLICATIONS

The present invention claims priority under 35 U.S.C. § 119 to Japanese Patent Application No. 2020-154220 filed on Sep. 15, 2020, the entire content of which is incorporated herein by reference.

### BACKGROUND

### Technological Field

The present disclosure relates to an X-ray dynamic image display apparatus, a storage medium, an X-ray dynamic image display method, and an X-ray dynamic image display system.

### Description of Related Art

In the medical field, image diagnosis has been performed on the basis of X-ray images or X-ray dynamic images that show regions of interest. For example, according to JP2016-34300A, a subject being imaged by X-ray imaging is also imaged with a video camera, and the obtained camera image and X-ray image are superposed for display.

### SUMMARY

In making a diagnosis on respiration, for example, a clinician observes not only images but also facial expressions and body movements during face-to-face interviews, visual inspections, and palpation, and subjectively judges therapeutic effects.

However, in the known art, the clinician cannot simultaneously evaluate external body movements and movements of internal organs. The clinician therefore may not identify causes of a disease or make objective evaluations on therapeutic effects, failing to provide effective medical care to the patient.

In treating diseases and injuries, rehabilitation often plays an important role. For example, according to Jun Ueki et al., “Statement on Respiratory Rehabilitation” Journal of the Japan Society for Respiratory Care and Rehabilitation, Vol. 27, No. 2, 2018, pp. 95-114, respiratory rehabilitation is a well-established therapeutic intervention with evidence that relieves dyspnea, anxiety and depression, and improves exercise tolerance and health-related quality of life and states of health. Examples of respiratory rehabilitation include pursed-lip breathing and abdominal breathing for relieving difficulty in breathing of a patient with chronic obstructive pulmonary disease (COPD). It is also known that a patient with a strong difficulty in breathing may breathe while moving muscles that do not move in normal breathing (e.g., shoulder).

There are, however, issues to be solved in rehabilitation. For example, when explaining how to do rehabilitation to a patient, a physiotherapist mainly explains it by words or by demonstrations so that the patient can imitate the demonstrations. The physiotherapist evaluates the effects of the rehabilitation by asking the patient about his/her condition. When, for example, the patient undergoes respiratory rehabilitation, the physiotherapist touches the patient with the hand to check the movement of the patient's chest.

Explanation on how to do rehabilitation and evaluation of rehabilitation effects are often subjective and depend on the skills of the physiotherapist. The patient may also find it difficult to keep motivated because the patient cannot objectively recognize the effects of rehabilitation. Accordingly, effective medical care may not be provided to the patient.

According to JP2016-34300A, the X-ray image and the camera moving image are superposed. This is for efficient positioning in X-ray imaging and may not allow doctors and physiotherapists to simultaneously evaluate external body movements and movements inside the body (e.g., organs). Therefore, JP2016-34300A may not contribute to providing effective medical care to the patient.

An object of the present invention is to provide effective medical care to patients.

To achieve at least one of the abovementioned objects, according to an aspect of the present invention, there is provided an X-ray-dynamic-image display apparatus including a first hardware processor that: obtains X-ray-dynamic-image related information on an X-ray dynamic image obtained through X-ray dynamic imaging and camera-moving-image related information on a camera moving image obtained through camera imaging; and synchronously displays the X-ray-dynamic-image related information and the camera-moving-image related information.

According to another aspect of the present invention, there is provided a non-transitory computer-readable storage medium storing a program that causes a computer to: obtain X-ray-dynamic-image related information on an X-ray dynamic image obtained through X-ray dynamic imaging and camera-moving-image related information on a camera moving image obtained through camera imaging; and synchronously display the X-ray-dynamic-image related information and the camera-moving-image related information.

According to another aspect of the present invention, there is provided an X-ray-dynamic-image display method including: obtaining X-ray-dynamic-image related information on an X-ray dynamic image obtained through X-ray dynamic imaging and camera-moving-image related information on a camera moving image obtained through camera imaging; and synchronously displaying the X-ray-dynamic-image related information and the camera-moving-image related information.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows an overall configuration of a dynamic image display system according to an embodiment of the present invention;

FIG. 2 shows an example position of a camera of FIG. 1;

FIG. 3 shows a flow of a sequence A for obtaining and displaying a dynamic image to be performed by the dynamic image display system of FIG. 1;

FIG. 4 shows an example of a dynamic analysis result when the region imaged in X-ray imaging is the chest;

FIG. 5 shows an example of a dynamic analysis image when the region imaged in X-ray imaging is the chest;

FIG. 6 shows an example of a camera-moving-image analysis image when the region imaged in X-ray imaging is the chest;

FIG. 7 shows an example of a camera-moving-image analysis image when the region imaged in X-ray imaging is the elbow;

FIG. 8 shows an example of a dynamic analysis result when the region imaged in X-ray imaging is a region related to swallowing;

FIG. 9 shows an example of a dynamic analysis image when the region imaged in X-ray imaging is a region related to swallowing;

FIG. 10 shows an example of a synchronous display screen displayed on a diagnosis console;

FIG. 11 shows an example of a synchronous display screen displayed on a mobile terminal;

FIG. 12 shows a flow of a sequence B for obtaining and displaying a dynamic image to be performed by the dynamic image display system of FIG. 1;

FIG. 13 is a flowchart of a process A for specifying the start of synchronous display to be performed by a controller of the diagnosis console in FIG. 1; and

FIG. 14 is a flowchart of a process B to be performed by the controller of the diagnosis console in FIG. 1 for specifying the start of synchronous display.

### DETAILED DESCRIPTION OF THE EMBODIMENTS

Hereinafter, an embodiment of the present invention is described with reference to the drawings. However, the scope of the present invention is not limited to the disclosed embodiment.

### First Embodiment

### [Configuration of Dynamic Image Display System 100]

First, a configuration in a first embodiment of the present invention is described.

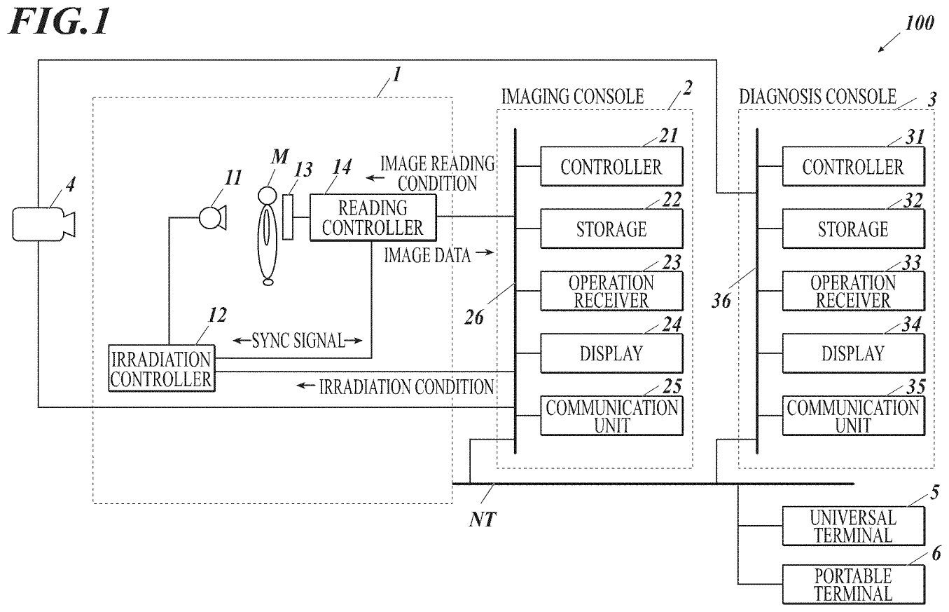

FIG. 1 shows a overall configuration of a dynamic image display system 100 in this embodiment.

As shown in FIG. 1, the dynamic image display system 100 includes: an imaging device 1; a camera 4; an imaging console 2; a diagnosis console 3; a universal terminal 5; and a portable terminal 6. The imaging device 1, the camera 4, and the imaging console 2 are connected via cables, for example. The imaging console 2, the diagnosis console 3, the universal terminal 5, and the portable terminal 6 can be connected over a communication network NT, such as a local area network (LAN). Among the components constituting the dynamic image display system 100, the imaging device 1, the imaging console 2, and the diagnosis console 3 conform to the digital image and communications in medicine (DICOM) standard and communicate with one another in accordance with the DICOM.

The dynamic image display system 100 performs X-ray imaging with the imaging device 1 to obtain an X-ray dynamic image that shows the dynamic state of the imaging part of a subject M. The dynamic image display system 100 also images (captures moving images), with the camera 4, a part of the subject M related to the imaging part, thereby obtaining a camera moving image that shows the dynamic state of the related part. The dynamic image display system 100 synchronously displays X-ray-dynamic-image related information on the obtained X-ray dynamic image and camera-moving-image related information on the obtained camera moving image. Thus, the dynamic image display system 100 supports diagnoses and rehabilitations to provide effective medical treatment to the patient.

The X-ray-dynamic-image related information includes at least the X-ray dynamic image or the dynamic analysis result obtained by analyzing the X-ray dynamic image. The X-ray-dynamic-image related information may include both the X-ray dynamic image and the dynamic analysis result. The X-ray dynamic image is obtained through X-ray dynamic imaging and is not yet dynamically analyzed. The X-ray dynamic image may be a dynamic image on which processing, such as general noise removal and edge processing, has been performed. The dynamic analysis result is obtained by performing dynamic analysis of the X-ray dynamic image, which is obtained through X-ray dynamic imaging. The dynamic analysis result includes, for example, a dynamic analysis image and information other than images, such as a graph and numerical values.

The camera-moving-image related information includes at least the camera moving image or a camera-moving-image analysis result obtained by analyzing the camera moving image. The camera-moving-image related information may include both the camera moving image and the camera-moving-image analysis result. The camera moving image is obtained by the camera 4 and is not yet subjected to image analysis. The camera moving image may be a moving image on which processing, such as general noise removal and edge processing, has been performed. The camera-moving-image analysis result is obtained by performing image analysis of the camera moving image obtained by the camera 4. The camera-moving-image analysis result may include the camera-moving-image analysis image and information other than images, such as a graph and numerical values. Examples of the camera-moving-image analysis result include results of shape analysis and angle analysis of the imaged part.

Further, “synchronous display” refers to displaying an item(s) of the X-ray-dynamic-image related information and an item(s) of the camera-moving-image related information that coincide in timing in imaging such that they are synchronize with each other. “Coincide in timing” may include some difference in timing and is not limited to exact coincidence. For example, when the X-ray-dynamic-image related information and the camera-moving-image related information are played as moving images, the dynamic image display system 100 may synchronously display the X-ray-dynamic-image related information and the camera-moving-image related information by aligning their start positions (positions in the time axis at which the play starts), display positions (frames), and/or phases.

[Configuration of Imaging Device 1]

The imaging device 1 performs X-ray dynamic imaging. X-ray dynamic imaging refers to continuously obtaining images of the subject M in motion to obtain an X-ray dynamic image that consists of multiple frame images (frames) and that shows the dynamic state of the subject M. The imaging device 1 obtains the X-ray dynamic image of the subject M by repetitively emitting pulsed radiation (X-rays) to the subject M at predetermined time intervals (pulse emission) or continuously emitting radiation to the subject M without a break at a low dose rate (continuous emission). In the embodiments described below, dynamic imaging is performed through pulse emission as an example.

A radiation source 11 is positioned to face a radiation detector 13 with the subject M inbetween. The radiation source 11 emits radiation (X-rays) to the subject M under the control of an irradiation controller 12.

The irradiation controller 12 is connected to the imaging console 2. The irradiation controller 12 controls the radiation source 11 and performs radiation imaging on the basis of irradiation conditions input by the imaging console 2. The irradiation conditions input by the imaging console 2 include, for example, the pulse rate, the pulse width, the pulse interval, the number of frames to be captured in one imaging, current values of an X-ray tube, voltage values of the X-ray tube, and a type of added filter. The pulse rate is the number of radiation emissions per second and matches the frame rate described below. The pulse width is a period of time for one radiation emission. The pulse interval is an interval between the start of one radiation emission and the start of the next radiation emission. The pulse interval matches the frame interval described below.

The radiation detector 13 is constituted of a semiconductor image sensor, such as a flat panel display (FPD). The FPD is constituted of detection elements (pixels) arranged at predetermined points on a substrate, such as a glass substrate, in a matrix. The detection elements detect radiation (intensity of radiation) that has been emitted from the radiation source 11 and passed through at least the subject M, convert the detected radiation into electric signals, and accumulate the electric signals therein. Each pixel includes a switch, such as a thin film transistor (TFT). Types of the FPD include an indirect conversion type that converts X-rays into electric signals with photoelectric conversion element(s) via scintillator(s) and a direct conversion type that directly converts X-rays into electric signals. Either type can be used.

The radiation detector 13 is positioned to face the radiation source 11 with the subject M inbetween.

A reading controller 14 is connected to the imaging console 2. The reading controller 14 controls the switches of the pixels of the radiation detector 13 and switches the pixels to read the electric signals accumulated in the pixels, on the basis of image reading conditions input by the imaging console 2. The reading controller 14 thus obtains image data. This image data is frame images. The reading controller 14 then outputs the obtained frame images to the imaging console 2. The image reading conditions include the frame rate, the frame interval, the pixel size, and the image size (matrix size). The frame rate is the number of frame images obtained per second. The frame rate matches the pulse rate. The frame interval is a period of time from the start of one frame image obtaining action to the start of the next frame image obtaining action. The frame interval matches the pulse interval.

The radiation emission controller 12 and the reading controller 14 are connected to each other so that they can exchange sync signals and synchronize the radiation emission operation and the image reading operation.

[Configuration of Imaging Console 2]

The imaging console 2 outputs the irradiation conditions and the image reading conditions to the imaging device 1 and controls X-ray emitting operation and radiographic-image reading operation of the imaging device 1. In the first embodiment, the imaging console 1 also controls the start and end of imaging of the camera 4.

The imaging console 2 includes, as shown in FIG. 1, a controller 21, a storage 22, an operation receiver 23, a display 24 and a communication unit 25. These components are connected via a bus 26.

The controller 21 includes a central processing unit (CPU) and a random access memory (RAM).

The CPU of the controller 21 reads a system program or various processing programs stored in the storage 22 in accordance with operations on the operation receiver 23, loads the read program into the RAM and, in accordance with the loaded programs, performs various processes, such as the process performed by the imaging console 2 in a sequence for obtaining and displaying a dynamic image. The CPU thus centrally controls operations of the components of the imaging console 2 and the radiation emission operation and the image reading operation of the imaging device 1. The controller 21 functions as synchronous imaging controller.

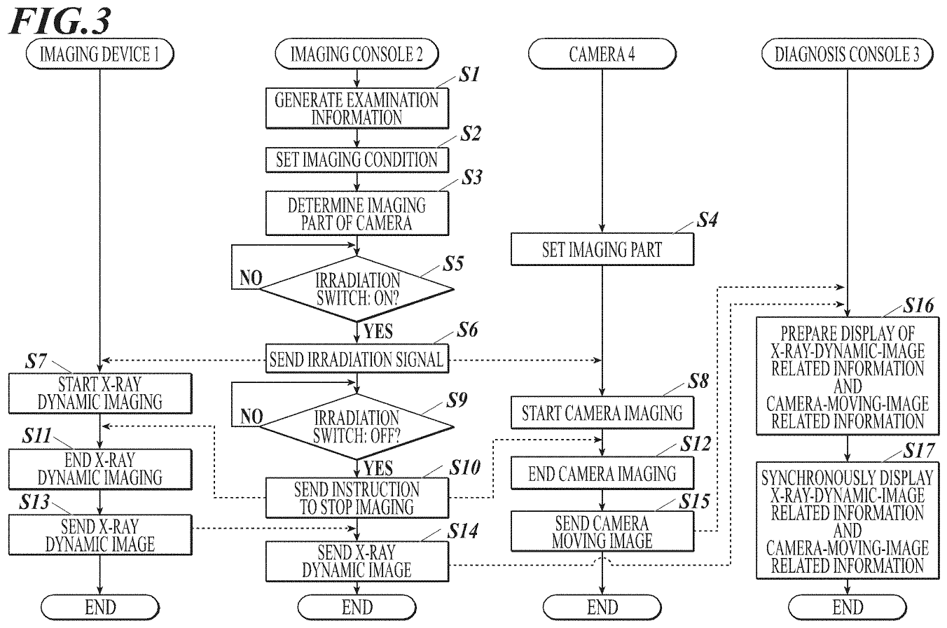

The storage 22 is constituted of a nonvolatile semiconductor memory and/or a hard disk, for example. The storage 22 stores various programs to be executed by the controller 21, parameters necessary for performing processes of the programs, data including process results, and so forth. For example, the storage 22 stores a program for the imaging console 2 to perform the process in the sequence for obtaining and displaying a dynamic image shown in FIG. 3. The storage 22 also stores the radiation emission conditions and the image reading conditions for respective imaging parts. The programs are stored in the form of computer-readable program codes. The controller 21 performs operations in accordance with the program codes.

The operation receiver 23 includes a keyboard including cursor keys, number input keys and various function keys, and a pointing device, such as a mouse. The operation receiver 23 outputs, to the controller 21, a command signal input by a key operation on the keyboard or by a mouse operation. The operation receiver 23 may include a touchscreen on the display screen of the display 24. In this case, the operation receiver 23 outputs command signals input via the touchscreen to the controller 21. The operation receiver 23 further includes an irradiation switch.

The display 24 is constituted of a monitor, such as a liquid crystal Display (LCD) or a cathode ray tube (CRT), and displays commands input by the operation receiver 23 and data in accordance with commands of display signals input by the controller 21.

The communication unit 25 includes a LAN adapter, a modem, and a terminal adapter (TA). The communication unit 25 controls data exchange with devices connected to the communication network NT.

[Configuration of Diagnosis Console 3]

The diagnosis console 3 (X-ray dynamic image display apparatus) obtains an X-ray dynamic image from the imaging console 2 and also obtains a camera moving image captured by the camera 4 during the X-ray dynamic imaging. The diagnosis console 3 then synchronously displays the X-ray-dynamic-image related information, which is related to the obtained X-ray dynamic image, and the camera-moving-image-related information, which is related to the obtained camera moving image. The diagnosis console 3 thus supports diagnoses by doctors and rehabilitation.

The diagnosis console 3 includes, as shown in FIG. 1, a controller 31 (hardware processor), a storage 32, an operation receiver 33, a display 34, and a communication unit 35. These components are connected via a bus 36.

The controller 31 includes a CPU and a RAM. The CPU of the controller 31 reads a system program and various process programs stored in the storage 32 in accordance with operations input with the operation receiver 33, loads the read programs in the RAM and, in accordance with the loaded programs, performs various processes, such as the process of the diagnosis console 3 in the sequence for obtaining and displaying a dynamic image described below. The controller 31 thus centrally controls operations of the components of the diagnosis console 3. The controller 31 functions as an obtaining unit and synchronous display unit.

The storage 32 is constituted of a nonvolatile semiconductor memory and/or a hard disk, for example. The storage 32 stores various programs for the controller 31 for executing various processes, parameters necessary for executing the processes, and data including processing results. For example, the storage 32 stores a program for the diagnosis console 3 to execute the process in the sequence for obtaining and displaying a dynamic image shown in FIG. 3. These programs are stored in the form of computer readable program codes, and the controller 31 appropriately performs operations in accordance with the program codes.

The operation receiver 33 includes a keyboard including cursor keys, number input keys and various function keys, and a pointing device, such as a mouse. The operation receiver 33 outputs, to the controller 31, a command signal input by a key operation on the keyboard or by a mouse operation. The operation receiver 33 may include a touchscreen on the display screen of the display 34. In this case, the operation receiver 33 outputs command signals input via the touchscreen to the controller 31.

The display 34 is constituted of a monitor, such as an LCD or a CRT, and displays various contents in accordance with commands of display signals input by the controller 31.

The communication unit 35 includes a LAN adapter, a modem, and a TA, and controls data exchange with devices connected over the communication network NT.

[Configuration of Camera 4]

The camera 4 consists of, for example, an optical camera, such as a charge coupled device (CCD) camera or a complementary metal oxide semiconductor device (CMOS) camera. The camera 4 performs video shooting of a part (region) of the subject M that is related to the imaging part in X-ray dynamic imaging. The camera 4 thus obtains a camera moving image constituted of multiple frame images. The camera 4 sends the camera moving image to the diagnosis console 3. The part related to the imaging part in X-ray dynamic imaging is, for example, the part the movement of which can affect the movement of the imaging part in X-ray dynamic imaging (the part that synchronizes with the imaging part in X-ray dynamic imaging).

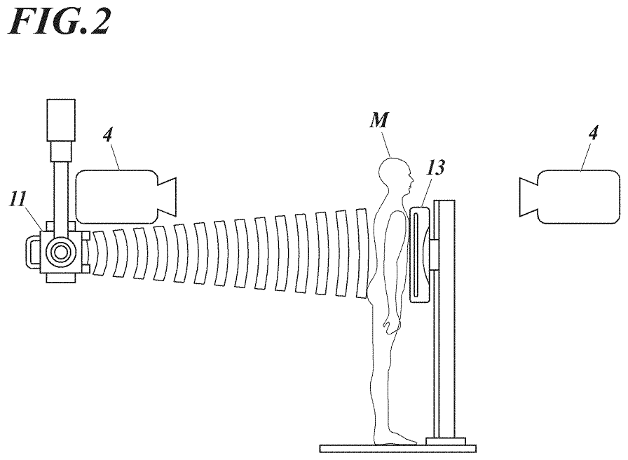

The position of the camera 4 is not limited to a specific position as long as the camera 4 can capture images of the part of the subject M related to the imaging part in X-ray dynamic imaging. For example, the camera 4 may be positioned close to the radiation source 11 as shown in FIG. 2, or may be positioned on the side of the radiation detector 13. There may be multiple cameras 4 as needed.

[Configuration of Universal Terminal 5 and Mobile Terminal 6]

The universal terminal 5 consists of, for example, a personal computer (PC) that a patient can use.

The universal terminal 5 displays the X-ray-dynamic-image related information and the camera-moving-image related information sent from the diagnosis console 3.

The mobile terminal 6 consists of, for example, a smartphone or a tablet that a patient can use. The mobile terminal 6 displays the X-ray-dynamic-image related information and the camera-moving-image related information sent from the diagnosis console 3.

[Configuration of Dynamic Image Display System 100]

Next, the operation of the dynamic image display system 100 in the first embodiment is described.

FIG. 3 shows a flow of a sequence for obtaining and displaying a dynamic image (referred to as a sequence A) performed by the dynamic image-display system 100. The flow of the sequence A is described with reference to FIG. 3.

First, when an imaging operator operates the operation receiver 23 of the imaging console 2 and inputs patient information, such as the name, height, weight, age, and sex, of the imaging target (subject M), the imaging console 2 generates examination information of the subject M (Step S1).

Next, the imaging console 2 reads irradiation conditions in the storage 22 corresponding to the imaging part and sets the conditions to the irradiation controller 12. The imaging console 2 also reads image reading conditions in the storage 22 and sets the conditions to the reading controller 14 (Step S2).

Next, the imaging part by the camera 4 is determined at the imaging console 2 (Step S3).

As described above, the imaging part by the camera 4 is related to the imaging part in X-ray dynamic imaging. For example, the imaging part by the camera 4 is a part the movement of which can affect the movement of the imaging part in X-ray dynamic imaging.

The storage 22 stores, for example, a table that associates the imaging parts in X-ray dynamic imaging and the imaging parts by the camera 4. In Step S3, the controller 21 refers to the table and determines the imaging part by the camera 4 on the basis of the imaging part in X-ray dynamic imaging.

When the imaging part in X-ray dynamic imaging is associated with multiple imaging parts by the camera 4 in the table, the multiple imaging parts by the camera 4 may be displayed on the display 24 so that the imaging operator can select and determine the imaging part by the camera 4 from the multiple parts.

In respiratory rehabilitation, a COPD patient performs, for example, “pursed-lip breathing” and “abdominal breathing” to relieve difficulty in breathing. It is also known that a patient with a strong difficulty in breathing may breathe while moving muscles that do not move in normal breathing (e.g., shoulder). With regards to the above, when the imaging part in X-ray dynamic imaging is the chest as an example, the candidate imaging part by the camera 4 may be a respiratory part, such as lips or the part corresponding to the irradiation field (chest).

For another example, when the imaging part in X-ray dynamic imaging is a part to be handled in orthopedics (e.g., part including a joint), the imaging part by the camera 4 may be a part related to the joint. For example, when the imaging part in X-ray dynamic imaging is an elbow joint or a knee joint, the imaging part by the camera 4 is the elbow or the knee.

For another example, when the imaging part in X-ray dynamic imaging is a part related to swallowing (i.e., examination of swallowing), the imaging part by the camera 4 is a part related to swallowing, such as the mouth in swallowing food containing a contrast medium (e.g., barium).

The determined imaging part by the camera 4 is displayed on the display 24, for example.

When the imaging part by the camera 4 is determined, the camera 4 receives the setting of the imaging part done by the user's operation (Step S4).

When the imaging part by the camera 4 is determined, the imaging operator positions the subject M between the radiation source 11 and the radiation detector 13. The imaging operator also does setting of the camera 4 so that the imaging region of the camera 4 includes the imaging part by the camera 4 determined in Step S3. When preparation for imaging is completed, the imaging operator presses the irradiation button.

The patient's imaging posture and imaging direction may be any of the following: standing PA (posterior to anterior) view, standing AP (anterior to posterior) view, sitting PA view, sitting AP view, and laying posture.

When the imaging console 2 detects that the irradiation button is pressed (the button is turned on) (Step S5: YES), the imaging console 2 sends irradiation signals to the irradiation controller 12, the reading controller 14, and the camera 4 (Step S6). In response to receiving the irradiation signals, the imaging device 1 starts X-ray dynamic imaging (Step S7) and the camera 4 starts capturing a moving image (camera imaging) (Step S8).

Thus, when the irradiation button is pressed, the controller 21 of the imaging console 2 sends the irradiation signals to the irradiation controller 12, the reading controller 14, and the camera 4 so that these devices synchronously start imaging.

In X-ray dynamic imaging, the radiation source 11 emits radiation at the pulse interval set in the irradiation controller 12, and the radiation detector 13 obtains frame images. To the obtained frame images, information on the imaging date and time is added as information related to synchronous display. For example, the information is written in the header region of image data in DICOM format.

When the imaging console 2 detects that the irradiation switch is turned off (Step S9: YES), the imaging console 2 sends imaging-end signals to the irradiation controller 12, the reading controller 14, and the camera 4 (Step S10), so that X-ray dynamic imaging and camera imaging end (Steps S11, S12).

The imaging device 1 sends the frame images constituting the X-ray dynamic image obtained in X-ray dynamic imaging to the imaging console 2 (Step S13). The imaging device 1 may successively send the frame images of the X-ray dynamic image to the imaging console 2 in order of being obtained by the radiation detector 13.

The imaging console 2 adds, to each of the frame images obtained from the imaging device 1, information such as an identification ID for identifying the X-ray dynamic image, patient information, the imaging part, the irradiation conditions, and the image reading conditions. The information is written in the header region of the image data in the DICOM format, for example. The imaging console 2 then sends the frame images to the diagnosis console 3 via the communication unit 25 (Step S14).

The camera 4 adds, to each of the frame images constituting the camera moving image, information related to synchronous display, and sends the camera moving image to the diagnosis console 3 (Step S15). The information related to synchronous display includes at least imaging date and time.

When receiving the X-ray dynamic image and the camera moving image via the communication unit 35, the diagnosis console 3 prepares displaying of X-ray-dynamic-image related information and camera-moving-image related information (Step S16).

As described above, the X-ray-dynamic-image related information includes at least either the X-ray dynamic image or the dynamic analysis result obtained by analyzing the X-ray dynamic image.

In displaying the dynamic analysis result as the X-ray-dynamic-image related information, the X-ray dynamic image is analyzed in Step S16 to obtain the dynamic analysis result (e.g., dynamic analysis image, graph, numerical values).

In displaying the X-ray dynamic image as the X-ray-dynamic-image related information, no processing may be performed in Step S16. Image processing, such as normal noise removal or edge processing, may be performed in Step S16.

The camera-moving-image related information includes at least either the camera moving image or the camera-moving-image analysis result obtained by analyzing the camera moving image. In displaying the camera-moving-image analysis result as the camera-moving-image related information, the camera moving image is analyzed in Step S16 to obtain the camera-moving-image analysis result (e.g., camera-moving-image analysis image, graph, values). In displaying the camera moving image as the camera-moving-image related information, no processing may be performed in Step S16. Image processing, such as normal noise removal or edge processing, may be performed in Step S16.

The user may select information items to be displayed as the X-ray-dynamic-image related information and the camera-moving-image related information by operating the operation receiver 33. Alternatively, the information items to be displayed may be set beforehand for the respective imaging parts in X-ray dynamic imaging and in camera imaging.

The method of analyzing the X-ray dynamic image and the camera moving image in Step S16 is not limited to a specific one and may be a known analysis method.

In an X-ray dynamic image showing the chest, the position of the diaphragm (distance between the apex of the lung and the diaphragm), the width of the thorax, and the area of the lung field change with respiration, for example. The pixel signal values in the lung field also change with respiration. More specifically, the concentration of the lung field region is low at the maximum expiratory level and high at the maximum inspiratory level. In view of the above, when the imaging part is the chest, the X-ray dynamic image is analyzed in Step S16 to obtain: information on movements of the diaphragm, the thorax, and the lung field; and dynamic analysis image (e.g., ventilation analysis image, blood flow analysis image).

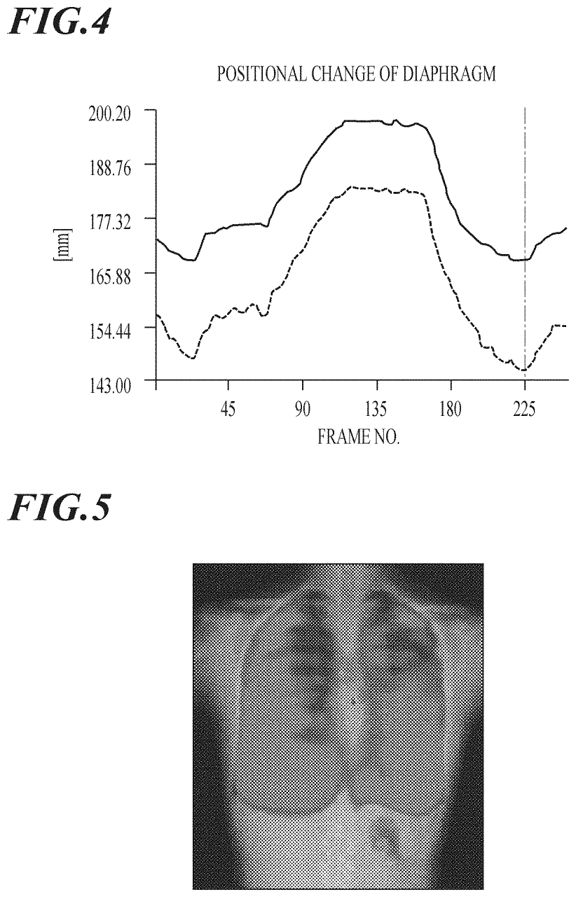

More specifically, the position of the diaphragm (distance between the apex of the lung and the diaphragm), the width of the thorax, and the area of the lung field are obtained for each frame image constituting the X-ray dynamic image. The diagnosis console 3 then obtains, for example, values and/or a graph(s) showing chronological changes of the obtained values as the dynamic analysis result. FIG. 4 shows an example of a graph showing chronological changes of the position of the diaphragm.

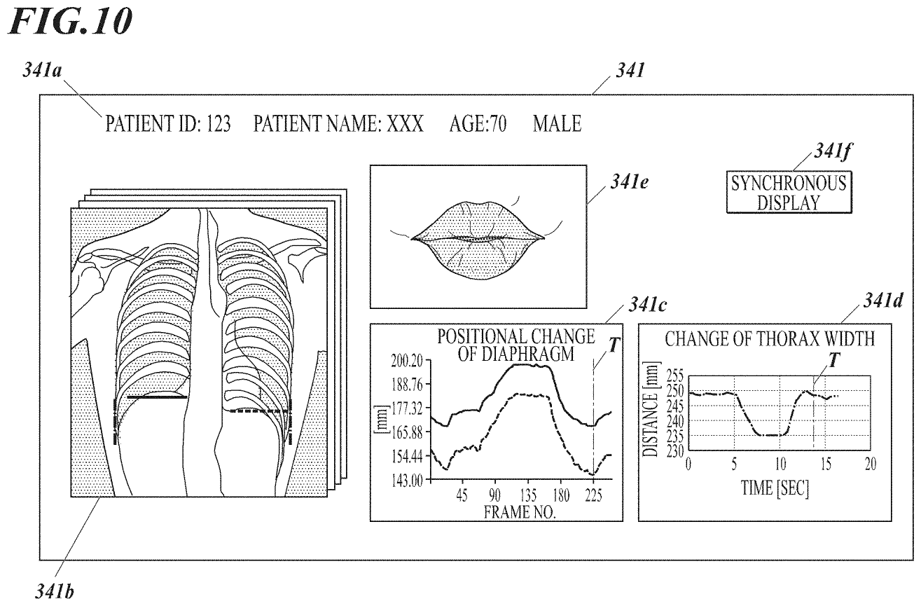

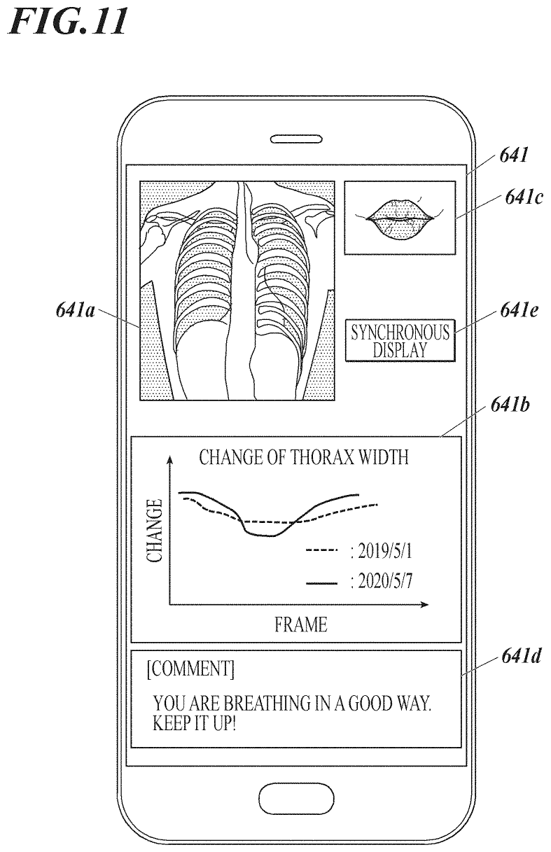

Instead, as shown in FIG. 10, a dynamic analysis image may be generated by superposing annotations indicating the position of the diaphragm and the width of the thorax on each frame image constituting the X-ray dynamic image.

Instead, the diagnosis console 3 may: extract a lung field region from each frame image constituting the X-ray dynamic image; divide the extracted lung field region into multiple small regions; associate the small regions among the frame images (e.g., associate the small regions that corresponds to the same position among the frame images); perform low-pass filtering in the time direction; and obtain differences in pixel signal values between adjacent frame images or differences in pixel signal values between the reference frame image and each frame image for the respective small regions. The diagnosis console 3 may then generate a dynamic analysis image (ventilation analysis image) in which colors corresponding to the obtained differences are layered on each frame image. FIG. 5 shows an example of the ventilation analysis image.

Instead, the diagnosis console 3 may: extract a lung field region from each frame image constituting the X-ray dynamic image; divide the extracted lung field region into multiple small regions; associate the small regions among the frame images (e.g., associate the small regions that correspond to the same position among the frame images); perform high-pass filtering in the time direction; and obtain differences in pixel signal values between adjacent frame images or differences in pixel signal values between the reference frame image and each frame image for the respective small regions. The diagnosis console 3 may then generate a dynamic analysis image (blood-flow analysis image) in which colors corresponding to the obtained differences are layered on each frame image.

When the imaging part by the camera 4, which has been captured during the X-ray dynamic imaging, is the lip, the diagnosis console 3 analyzes the camera moving image to obtain the camera-moving-image analysis image. The camera-moving-image analysis image shows, for example, information on the shape of the lip, information on the angles of the corners of the lip, and the shape and the corners of the lip.

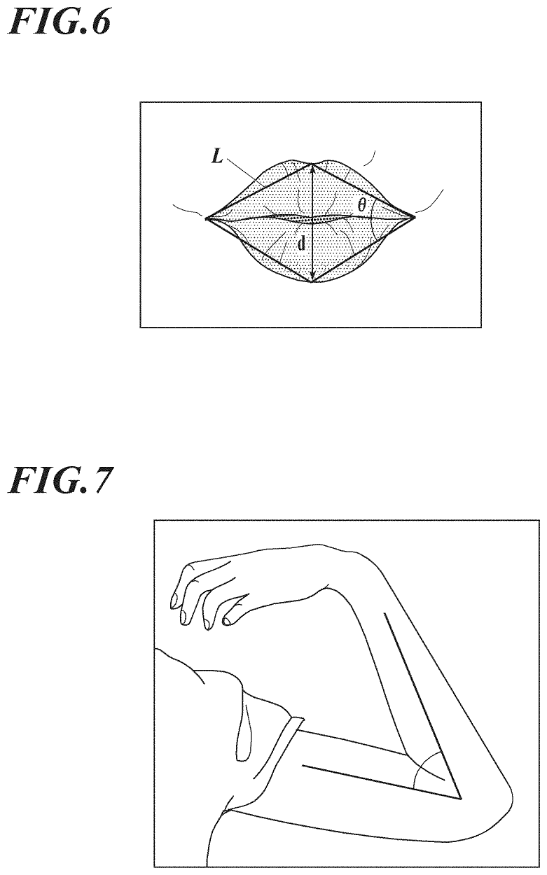

To generate the camera-moving-image analysis image, for example, the diagnosis console 3 recognizes the lip in each frame image constituting the camera moving image and connects the right and left corners of the mouth and the upper and lower apexes of the lips to generate lip shape information L. The diagnosis console 3 superposes the lip shape information L on each frame image of the camera moving image to generate the camera-moving-image analysis image. FIG. 6 shows an example of the camera-moving-image analysis image that includes the lip shape information L. Instead, the diagnosis console 3 may obtain the angle θ of the corner of the mouth and the distance D between the apexes of the upper and lower lips as the camera-moving-image analysis result. Instead, the diagnosis console 3 may obtain a graph showing chronological changes of the angle θ of the corner of the mouth and the distance D between the apexes of the upper and lower lips as the camera-moving-image analysis result.

When the imaging part in X-ray dynamic image is a joint, the diagnosis console 3 measures the angle of the joint in each frame image constituting the X-ray dynamic image, and obtains the angle values of the joint and/or a graph showing chronological changes of the angle values of the joint as the dynamic analysis result. The diagnosis console 3 may generate a dynamic analysis image by superposing, on each frame image constituting the X-ray dynamic image, annotations indicating the angles of the joint.

The same applies to the camera moving image. More specifically, when the imaging part by the camera 4 is a joint, the diagnosis console 3 measures the angle of the joint in each frame image constituting the camera moving image, and obtains the angle values of the joint and/or a graph showing chronological changes of the angle values of the joint as the camera-moving-image analysis result. The diagnosis console 3 may generate a camera-moving-image analysis image by superposing, on each frame image constituting the camera moving image, annotations indicating the angles of the joint (see FIG. 7).

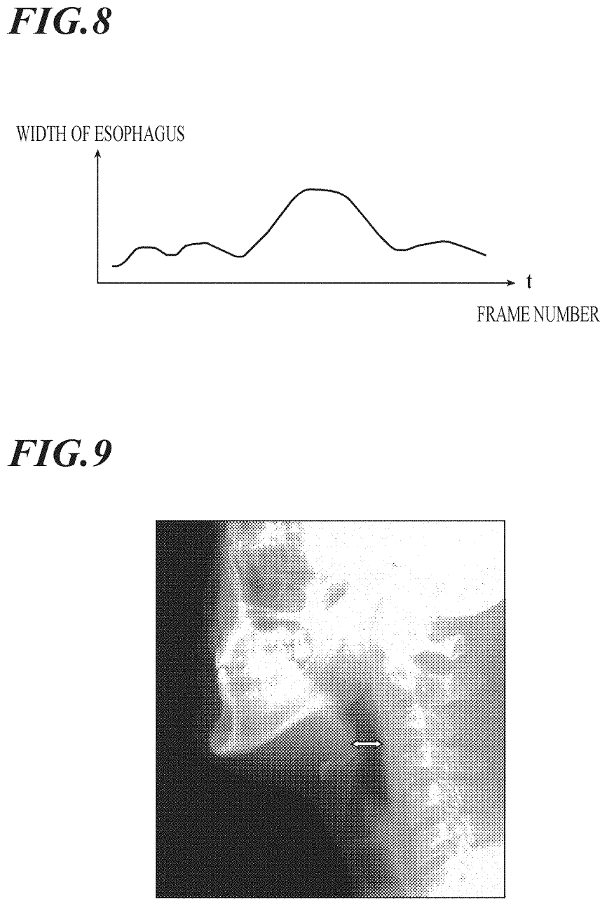

When the imaging part in X-ray dynamic imaging is related to swallowing (e.g., part including the mouth and the esophagus in swallowing food containing a contrast medium (e.g., barium)), the diagnosis console 3 recognizes the esophagus and measures the width of the esophagus in each frame image constituting the X-ray dynamic image. The diagnosis console 3 obtains the width of the esophagus and/or a graph showing chronological changes of the width of the esophagus (see FIG. 8) as the dynamic analysis result. As shown in FIG. 9, the diagnosis console 3 may generate the dynamic analysis image by superposing, on each frame image constituting the X-ray dynamic image, an annotation (arrow in FIG. 9) indicating the width of the esophagus.

When the imaging part by the camera 4 is the mouth in swallowing food containing a contrast medium (e.g., barium), the diagnosis console 3 measures the vertical length of the mouth (opening of the mouth) in each frame image constituting the camera moving image and obtains the vertical lengths and/or a graph showing chronological changes of the vertical length as the camera-moving-image analysis result. The diagnosis console 2 may generate a camera-moving-image analysis image by superposing, on each frame image constituting the camera moving image, an annotation indicating the vertical length of the mouth.

When the diagnosis console 3 is ready for display, the X-ray-dynamic-image related information and the camera-moving-image related information are synchronously displayed on the display 34 (Step S17). The sequence A for obtaining and displaying the dynamic image ends.

In Step S17, the diagnosis console 3 displays information items of the X-ray-dynamic-image related information and the camera-moving-image related information together, the information items corresponding to the same timing in imaging. “The same timing” may not refer to the exactly same timing and may have a small difference.

As described above, in the sequence A for obtaining and displaying the dynamic image, the imaging console 2 performs control such that X-ray dynamic imaging and camera imaging start in response to the irradiation switch being pressed (turned on) and that X-ray dynamic imaging and camera imaging end in response to the irradiation switch being turned off. That is, the period during which the X-ray dynamic image is captured is the same as the period during which the camera moving image is captured. Accordingly, when the X-ray-dynamic-image related information and the camera-moving-image related information are moving images captured at the same frame rate, the controller 31 can perform synchronous display by starting displaying the moving images (and/or the analysis results for the respective frame images) at the same time and switching the frame images at the same speed. When the two moving images are not captured at the same frame rate, the controller 31 adjusts timings of displaying the moving images such that the information items on the frame images corresponding to the same timing in imaging are displayed at the same time. For example, when one of the moving images is captured at 15 frames/second and the other moving image is captured at 30 frames/second, the controller 31 controls display such that the frame images captured at 30 frames/second are switched at double the speed of the moving image captured at 15 frames/second.

When the X-ray-dynamic-image related information and/or the camera-moving-image related information to be displayed are graphs, the controller 31 moves a mark on the graph (e.g., T in FIG. 10) at the speed corresponding to the frame rate in imaging, as with the above-described switching of frame images. When the X-ray-dynamic-image related information and/or the camera-moving-image related information are numerical values, the controller 31 switches the displayed numerical values at the speed corresponding to the frame rates, as with the above-described switching of frame images.

FIG. 10 shows an example of a synchronous display screen 341 displayed in Step S17. As shown in FIG. 10, the synchronous display screen 341 shows: patient information 341a, such as patient ID, patient name, and age; X-ray-dynamic-image related information 341b (dynamic analysis image showing the position of the diaphragm and the width of the thorax); X-ray-dynamic-image related information 341c (graph showing chronological changes of the position of the diaphragm); X-ray-dynamic-image related information 341d (graph showing chronological changes of the width of the thorax); camera-moving-image related information 341e (camera moving image of pursed-lip breathing); and the synchronous display button 341f. Regarding the X-ray-dynamic-image related information and the camera-moving-image related information, information on the start of the synchronous display is shown, for example. When the synchronous display button 341f is pressed, the diagnosis console 3 starts synchronously displaying the X-ray-dynamic-image related information and the camera-moving-image related information.

Thus, the camera-moving-image related information (moving image of pursed-lip breathing in FIG. 10) and X-ray-dynamic-image related information (diaphragm position and width of thorax shown in FIG. 10) are displayed synchronously. This allows doctors/physiotherapists to simultaneously observe the external body movement (movement of the lip during pursed-lip breathing shown in FIG. 10) and the internal movement (movement of the diaphragm and thorax shown in FIG. 10) and objectively evaluate effects of medical treatment and respiratory rehabilitation. Accordingly, effective treatment can be provided to the patient.

In Step S17, X-ray-dynamic-image related information and/or camera-moving-image related information in the past may also be displayed so that a doctor/physiotherapist can check chronological changes.

The controller 31 of the diagnosis console 3 may send the synchronous display screen, which is for synchronously displaying the X-ray-dynamic-image related information and the camera-moving-image related information, to the patient's universal terminal 5 or the mobile terminal 6 via the communication unit 35. In the case, the controller 31 may receive input of comments by co-medical staff (e.g., doctor, physiotherapist) and generate the synchronous display screen including the comment information and send the synchronous display screen to the universal terminal 5 or the mobile terminal 6.

FIG. 11 shows an example of a synchronous display screen 641 sent to the mobile terminal 6 and displayed on the terminal 6. In FIG. 11, the synchronous display screen 641 shows: X-ray-dynamic-image related information 641a (X-ray dynamic image of the chest); X-ray-dynamic-image related information 641b (graph of chronological changes of thorax width); camera-moving-image related information 641c (camera moving image); comments 641d by co-medical staff (e.g., doctor, physiotherapist); and a synchronous display button 641e. The X-ray-dynamic-image related information 641b (graph of chronological changes of thorax width) shows chronological changes of the current thorax width and the past thorax width together. Regarding the X-ray-dynamic-image related information and the camera-moving-image related information, information on the start of the synchronous display is shown, for example. When the synchronous display button 641e is pressed, the mobile terminal 6 starts synchronously displaying the X-ray-dynamic-image related information and the camera-moving-image related information.

Thus, the camera-moving-image related information (moving image of pursed-lip breathing in FIG. 11) and X-ray-dynamic-image related information (chest X-ray dynamic image and thorax width in FIG. 11) are displayed synchronously. This allows the user (patient) to simultaneously observe the external body movement (movement of the lip during pursed-lip breathing in FIG. 11) and the internal movement (movement of the lung field and thorax in FIG. 11) to objectively evaluate effects of medical treatment and rehabilitation. When there is an improvement in the state of the diseased part, the patient can objectively recognize the improvement and can be motivated. On the other hand, when there is an problem in doing rehabilitation (there is not an improvement), a co-medical staff (e.g., doctor or physiotherapist) can explain how to improve movements verbally or with comments by pointing out the movements that may not improve the state, on the basis of the X-ray-dynamic-image related information and the camera-moving-image related information. Thus, the co-medical staff can make explanations in an objective and easy-to-understand way to the patient. The co-medical staff can show the movements that improve the internal movements of the diseased part (or the movements that do not contribute improvements) in an objective way. Thus, effective medical treatment can be provided to the patient.

### Second Embodiment

The second embodiment of the present invention is described below.

In the first embodiment, X-ray dynamic imaging and camera imaging start in response to the irradiation switch being pressed (turned on), and X-ray dynamic imaging and camera imaging end in response to the irradiation switch being released (turned off). In the second embodiment, a camera switch is separately provided from the irradiation switch for instructing the camera 4 to start imaging.

The camera switch in the second embodiment is connected to the camera 4. The camera switch in the second embodiment is positioned close to the irradiation switch so that the imaging operator can press the irradiation switch and the camera switch at the same time.

Other components of the dynamic image display system 100 in the second embodiment are the same as those described in the first embodiment and are not described here. Hereinafter, the operation of the dynamic image display system 100 in the second embodiment is described.

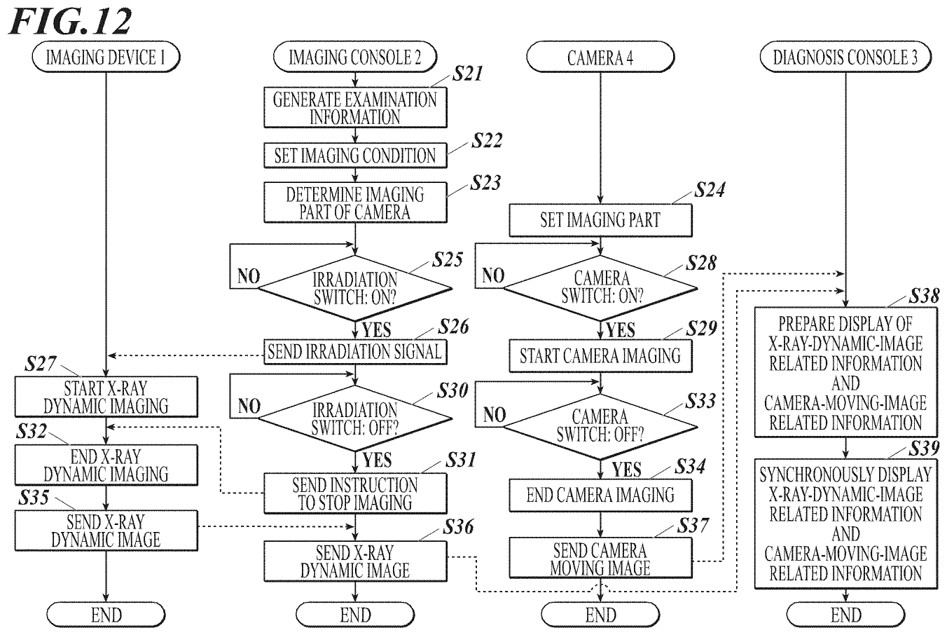

FIG. 12 shows a flow of a sequence for obtaining and displaying a dynamic image (referred to as sequence B) performed by the dynamic image-display system 100 in the second embodiment. The flow of the sequence B is described with reference to FIG. 12.

First, when an imaging operator operates the operation receiver 23 of the imaging console 2 to input patient information, such as the name, height, weight, age, and gender of the imaging target (subject M), the imaging console 2 generates examination information of the subject M (Step S21).

Next, the imaging console 2 reads irradiation conditions in the storage 22 corresponding to the imaging part and sets the conditions to the irradiation controller 12. The imaging console 2 also reads image reading conditions in the storage 22 and sets the conditions to the reading controller 14 (Step S22).

Next, the imaging part by the camera 4 is determined at the imaging console 2 (Step S23).

Step S23 is the same as the above-described Step S3 in FIG. 3 and is not described here.

The camera 4 receives the setting of the imaging part done by the user's operation (Step S24).

When the imaging part by the camera 4 is determined, the imaging operator positions the subject M between the radiation source 11 and the radiation detector 13. The imaging operator also does setting of the camera 4 so that the imaging region of the camera 4 includes the imaging part by the camera 4 determined in Step S23. When preparation for imaging is completed, the imaging operator presses the irradiation switch and the camera switch at the same time.

When the imaging console 2 detects that the irradiation button is pressed (turned on) (Step S25: YES), the imaging console 2 sends irradiation signals to the irradiation controller 12 and the reading controller 14 to start X-ray dynamic imaging (Step S27).

When the camera 4 detects that the camera switch is pressed (turned on) (Step S28; YES), the camera 4 starts capturing a moving image (camera imaging) (Step S29).

At the timing to end imaging, the imaging operator releases (turns off) the irradiation switch and the camera switch at the same time.

When the imaging console 2 detects that the irradiation switch is turned off (Step S30: YES), the imaging console 2 sends imaging-end signals to the irradiation controller 12 and the reading controller 14 (Step S31). Then X-ray dynamic imaging ends (Steps S32).

When the camera 4 detects that the camera switch is turned off (Step S33; YES), camera imaging ends (Step S34).

The imaging device 1 sends frame images constituting the X-ray dynamic image obtained in X-ray dynamic imaging to the imaging console 2 (Step S35). The imaging device 1 may successively send the frame images of the X-ray dynamic image to the imaging console 2 in order of being obtained by the radiation detector 13.

After the imaging ends, the imaging console 2 adds, to each of the frame images obtained from the reading controller 14, information such as an identification ID for identifying the X-ray dynamic image, patient information, imaging part, irradiation conditions, and image reading conditions. The information is written in the header region of the image data in the DICOM format, for example. The imaging console 2 then sends the frame images to the diagnosis console 3 via the communication unit 25 (Step S36).

After the imaging ends, the camera 4 adds, to each of the frame images constituting the obtained camera moving image (moving image), information related to synchronous display that includes at least imaging date and time, and sends the camera moving image to the diagnosis console 3 (Step S37).

When receiving the X-ray dynamic image and the camera moving image via the communication unit 35, the diagnosis console 3 prepares display of X-ray-dynamic-image related information and camera-moving-image related information (Step S38).

The process in Step S38 is the same as that in Step S16 in FIG. 3, and the description thereof is omitted.

When the diagnosis console 3 is ready for display, the X-ray-dynamic-image related information and the camera-moving-image related information are synchronously displayed on the display 34 (Step S39), and the sequence B for obtaining and displaying the dynamic image ends.

Step S39 is the same as Step S17 in FIG. 3 and is not described here.

As described above, in the second embodiment, the X-ray dynamic image and the camera moving image can be captured at the same timing with the irradiation switch and the camera switch for instructing imaging to the camera 4. Accordingly, the X-ray-dynamic-image related information and the camera-moving-image related information can be easily synchronously displayed. Therefore, as with the first embodiment, effective medical treatment can be provided to the patient.

### Third Embodiment

The third embodiment of the present invention is described below.

In the first and second embodiments, X-ray dynamic imaging and camera imaging are performed at the same time, and accordingly, the X-ray dynamic image and the camera moving image are captured for the same period of time. In the third embodiment, the diagnosis console 3 performs control such that the X-ray dynamic image and the camera moving image captured for the same period of time are specified and synchronously displayed.

The components of the dynamic image display system 100 in the third embodiment are the same as those described in the second embodiment and are not described here. Hereinafter, the operation of the dynamic image display system 100 in the third embodiment is described.

The sequence for obtaining and displaying a dynamic image in the third embodiment is substantially the same as the sequence in FIG. 12 in the second embodiment. In the third embodiment, the irradiation switch and the camera switch may not be pressed or released at the same time as long as the camera moving image during X-ray dynamic imaging is obtained. For example, camera imaging may start first, and X-ray dynamic imaging may be performed during camera imaging. In Step 38 for display preparation, the controller 31 performs a process for specifying start of synchronous display described below as well as performing image processing and analysis processing. The controller 31 thus specifies the frame image in the camera moving image that is captured at the time of starting X-ray dynamic imaging. In performing synchronous display in Step S39, the diagnosis console 3 synchronously displays the X-ray-dynamic-image related information and the camera-moving-image related information on the basis of the result of the process for specifying the start of synchronous display.

The process for specifying the start of synchronous display may be any of the processes A to C described below.

(Process A for specifying the start of synchronous display)

FIG. 13 shows a flowchart of the process A for specifying the start of synchronous display. The process A is performed by the controller 31 of the diagnosis console 3. Hereinafter, the process A is described with reference to FIG. 13.

First, the controller 13 obtains the time when X-ray dynamic imaging started from the information added to the first frame image in the X-ray dynamic image (Step S41).

Next, the controller 31 searches, in the frame images of the camera moving image, a frame image that was captured at the time when X-ray dynamic imaging started, on the basis of the information added to the camera moving image (Step S42). The frame image found in the search is specified as the start of the camera moving image in synchronous display, and the frame number thereof is stored in the RAM or the like (Step S43). The process A for specifying the start of synchronous display ends.

In synchronous display of the X-ray-dynamic-image related information and the camera-moving-image related information, the start of the X-ray-dynamic-image related information is the information on the first frame image of the X-ray dynamic image, and the start of the camera-moving-image related information is the information on the frame image of the camera moving image specified in Step S43. When the frame rate of the X-ray dynamic image is the same as the frame rate of the camera moving image, the synchronous display is performed by switching the X-ray-dynamic-image related information items and the camera-moving-image related information items at the same timing. When the frame rates are different, the controller 31 controls display such that an item in the X-ray-dynamic-image related information and an item in the camera-moving-image related information that correspond to the same timing in imaging are displayed at the same timing.

(Process B for Specifying the Start of Synchronous Display)

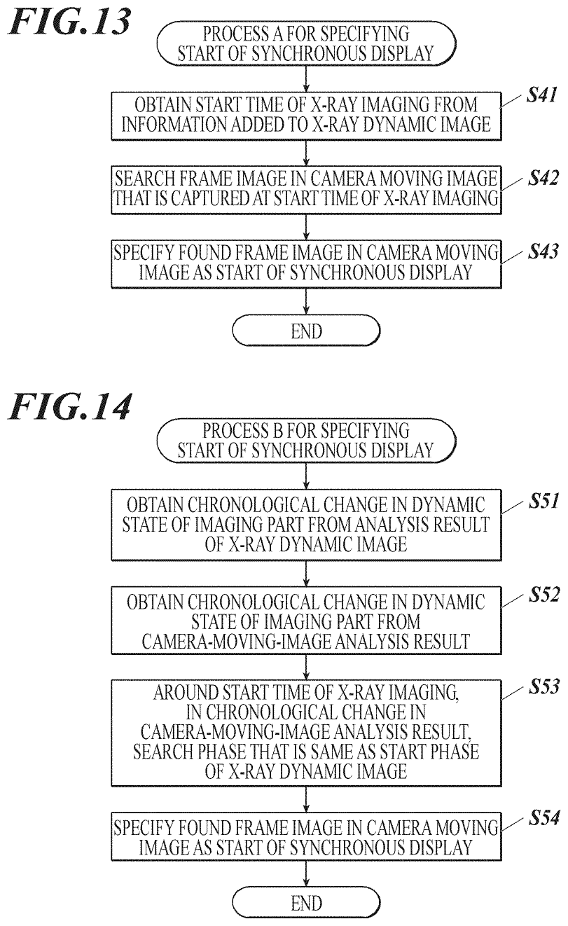

FIG. 14 shows a flowchart of the process B for specifying the start of synchronous display. The process B is performed by the controller 31 of the diagnosis console 3. Hereinafter, the process B is described with reference to FIG. 14.

First, the controller 31 obtains the chronological change of the dynamic state of the imaging part from the dynamic analysis result of the X-ray dynamic image (Step S51). When the imaging part is the chest, the chronological change of the dynamic state of the imaging part is, for example, the chronological change of the position of the diaphragm, chronological change of the thorax width, or chronological change of the lung field area.

The controller 31 obtains the chronological change of the dynamic state of the imaging part from the camera-moving-image analysis result (Step S52). When the imaging part by the camera 4 is the lip, the chronological change of the imaging part by the camera 4 is, for example, the chronological change in the angle of the corner of the mouth.

Next, in the chronological change of the dynamic state of the imaging part in the X-ray dynamic image, the controller 31 obtains the phase at the start of X-ray dynamic imaging (start phase of the X-ray dynamic image). The controller 31 then searches, around the start time of X-ray dynamic imaging, in the chronological change of the imaging part in the camera moving image, the frame image that shows the same phase as the start phase of X-ray dynamic image (Step S53).

Herein, the dynamic state of the imaging part in the camera moving image synchronizes with the dynamic state of the imaging part in the X-ray dynamic image (the movement of the imaging part in the camera moving image affects the movement of the imaging part in the X-ray dynamic image). Therefore, cycles in chronological changes of these two dynamic states are the same. That is, the phase (position in a cycle) at a certain timing of the imaging part in the camera moving image should be the same as the phase at the same timing of the imaging part in the X-ray dynamic image. The controller 31 searches, around the start time of X-ray dynamic imaging, in the chronological change of the imaging part in the camera moving image, the frame image that shows the same phase as the start phase of the X-ray dynamic image.

The controller 31 specifies the frame image found in Step S53 as the start frame image of the camera moving image in synchronous display, and stores the frame number of the start frame image in the RAM or the like (Step S54). The process B for specifying the start of synchronous display ends.

In synchronous display of the X-ray-dynamic-image related information and the camera-moving-image related information, the start of the X-ray-dynamic-image related information is the information on the first frame image of the X-ray dynamic image, and the start of the camera-moving-image related information is the information on the frame image of the camera moving image specified in Step S54. When the frame rate of the X-ray dynamic image is the same as the frame rate of the camera moving image, the synchronous display is performed by switching the X-ray-dynamic-image related information items and the camera-moving-image related information items at the same timing. When the frame rates are different, the controller 31 controls display such that an item in the X-ray-dynamic-image related information and an item in the camera-moving-image related information that correspond to the same timing in imaging are displayed at the same timing.

(Process C for Specifying the Start of Synchronous Display)

In the processes A and B, the controller 31 of the diagnosis console 3 automatically specifies the start frame images of synchronous display. In the process C, the diagnosis console 3 receives inputs of information on synchronous display (herein, information on the start frame image of synchronous display) by the user. On the basis of the input information, the diagnosis console 3 performs synchronous display.

In the process C, the controller 31 of the diagnosis console 3 causes the display 34 to display a start selection window (not illustrated). The start selection window shows, for example, thumbnail images of a series of frame images constituting the X-ray dynamic image and thumbnail images of a series of frame images constituting the camera moving image along the time axis. In the start selection window, when the user selects, with the operation receiver 33 (e.g., mouse), frame images in the X-ray dynamic image and the camera moving image as the start of synchronous display, the controller 31 sets the selected frame images as the start frame images of synchronous display. On the basis of the set start frame images, the diagnosis console 3 synchronously displays the X-ray-dynamic-image related information and the camera-moving-image related information. Alternatively, in the start selection window, the user may specify, with the operation receiver 33 (e.g., mouse), a group of the frame images to be synchronously displayed. The diagnosis console 3 may synchronously display the X-ray-dynamic-image related information and the camera-moving-image related information corresponding to the group of frame images specified by the user. The process C may be performed in response to the user's operation to adjust the result of the automatic process A or B.

According to the third embodiment, the X-ray-dynamic-image related information and the camera-moving-image related information can be synchronously displayed even when X-ray dynamic imaging and camera imaging are not performed synchronously.

As described above, the controller 31 of the diagnosis console 3 (claim 1)

This allows a doctor/physiotherapist to simultaneously observe the internal and external movements of the body and objectively evaluate effects of medical treatment or rehabilitation. Accordingly, the doctor/physiotherapist can provide effective medical treatment to the patient.

For example, assume that the imaging part in the X-ray dynamic image is the chest and the imaging part in the camera moving image is related to respiration. By synchronously displaying the camera moving image and the X-ray dynamic image, the physiotherapist can explain respiration actions to the patient from objective viewpoints. Further, by synchronously displaying the camera moving image and the dynamic analysis result, the physiotherapist/doctor can explain effects of respiratory rehabilitation to the patient from objective viewpoints. The physiotherapist thus can provide more effective rehabilitation to the patient.

For another example, assume that the imaging part in the X-ray dynamic image is the part related to orthopedics and the imaging part in the camera moving image is related to orthopedics. By synchronously displaying the camera moving image and the X-ray dynamic image, the physiotherapist can explain movements of a joint to the patient from objective viewpoints. Further, by synchronously displaying the camera moving image and the X-ray dynamic analysis result, the physiotherapist can explain effects of medical treatment on the joint to the patient from objective viewpoints. The physiotherapist thus can provide more effective rehabilitation to the patient.

For another example, assume that the imaging parts in the X-ray dynamic image and the camera moving image are both related to swallowing. By synchronously displaying the camera moving image and the X-ray dynamic image, the physiotherapist can explain movements of the mouth to the patient from objective viewpoints. Further, by synchronously displaying the camera moving image and the X-ray dynamic analysis result, the physiotherapist can explain effects of medical treatment on swallowing to the patient. The physiotherapist thus can provide more effective rehabilitation to the patient.

The above-described embodiments are preferred examples of the dynamic image display system of the present invention and not intended to limit the present invention.

For example, in the first to third embodiments, the diagnosis console 3 specifies the start frame image for synchronous display and displays the X-ray-dynamic-image related information items and the camera-moving-image related information items that correspond to the specified start frame image and the following frame images. However, all of the X-ray-dynamic-image related information items and the camera-moving-image related information items that correspond to the start frame image and the following frame images may not be synchronized. For example, when the imaging part is the joint part, the X-ray-dynamic-image related information items and the camera-moving-image related information items that correspond to the fully extended joint and fully bended joint may be extracted and synchronously displayed.

Further, the dynamic analysis result and the camera-moving-image analysis result described in the above embodiments are examples and not limited to the embodiments.

Further, in the above description, as a computer-readable medium storing the program of the present invention, a hard disk and/or a nonvolatile semiconductor memory is used. However, the computer-readable medium is not limited to these examples. As the computer readable medium, a portable storage medium, such as a CD-ROM, can also be used.

Further, as a medium to provide data of the program of the present invention, a carrier wave can be used.

Detailed configurations and detailed operations of the components of the dynamic image display system 100 can also be appropriately modified without departing from the scope of the present invention.

What is claimed is:

1. An X-ray-dynamic-image display apparatus comprising a first hardware processor that:
obtains X-ray-dynamic-image related information on an X-ray dynamic image obtained through X-ray dynamic imaging and camera-moving-image related information on a camera moving image obtained through camera imaging; and
synchronously displays the X-ray-dynamic-image related information and the camera-moving-image related information.

2. The X-ray-dynamic-image display apparatus according to claim 1, wherein
the X-ray-dynamic-image related information includes at least the X-ray dynamic image or a dynamic analysis result that is obtained by analyzing the X-ray dynamic image.

3. The X-ray-dynamic-image display apparatus according to claim 2, wherein the dynamic analysis result is a dynamic analysis image.

4. The X-ray-dynamic-image display apparatus according to claim 2, wherein the X-ray-dynamic-image related information includes both the X-ray dynamic image and the dynamic analysis result.

5. The X-ray-dynamic-image display apparatus according to claim 1, wherein the camera-moving-image related information includes at least the camera moving image or a camera-moving-image analysis result that is obtained by analyzing the camera moving image.

6. The X-ray-dynamic-image display apparatus according to claim 5, wherein the camera-moving-image analysis result is a camera-moving-image analysis image.

7. The X-ray-dynamic-image display apparatus according to claim 5, wherein the camera-moving-image related information includes both the camera moving image and the camera-moving-image analysis result.

8. The X-ray-dynamic-image display apparatus according to claim 1, wherein the camera moving image shows a part of a subject that relates to a part of the subject shown in the X-ray dynamic image.

9. The X-ray-dynamic-image display apparatus according to claim 8, wherein the part shown in the camera moving image is a part that relates to respiration.

10. The X-ray-dynamic-image display apparatus according to claim 8, wherein the part shown in the camera moving image is a part that relates to orthopedics or a part that relates to swallowing.

11. The X-ray-dynamic-image display apparatus according to claim 1, wherein
the X-ray-dynamic-image related information and the camera-moving-image related information both include synchronous display information for synchronously displaying the X-ray-dynamic-image related information and the camera-moving-image related information, and
the first hardware processor synchronously displays the X-ray-dynamic-image related information and the camera-moving-image related information based on the synchronous display information.

12. The X-ray-dynamic-image display apparatus according to claim 1, further comprising an information receiver that receives synchronous display information, wherein
the first hardware processor synchronously displays the X-ray-dynamic-image related information and the camera-moving-image related information based on the received synchronous display information.

13. The X-ray-dynamic-image display apparatus according to claim 1, wherein
the X-ray dynamic imaging and the camera imaging are synchronously performed by a second hardware processor, and
the first hardware processor synchronously displays the X-ray-dynamic-image related information and the camera-moving-image related information based on the X-ray dynamic imaging and the camera imaging that are synchronously performed by the second hardware processor.

14. A non-transitory computer-readable storage medium storing a program that causes a computer to:
obtain X-ray-dynamic-image related information on an X-ray dynamic image obtained through X-ray dynamic imaging and camera-moving-image related information on a camera moving image obtained through camera imaging; and
synchronously display the X-ray-dynamic-image related information and the camera-moving-image related information.

15. The storage medium according to claim 14, wherein the X-ray-dynamic-image related information includes at least the X-ray dynamic image or a dynamic analysis result that is obtained by analyzing the X-ray dynamic image.

16. The storage medium according to claim 15, wherein the dynamic analysis result is a dynamic analysis image.

17. The storage medium according to claim 14, wherein the camera-moving-image related information includes at least the camera moving image or a camera-moving-image analysis result that is obtained by analyzing the camera moving image.

18. The storage medium according to claim 17, wherein the camera-moving-image analysis result is a camera-moving-image analysis image.

19. The storage medium according to claim 14, wherein the camera moving image shows a part of a subject that relates to a part of the subject shown in the X-ray dynamic image.

20. The storage medium according to claim 19, wherein the part shown in the camera moving image is a part that relates to respiration.

21. An X-ray-dynamic-image display method comprising:
obtaining X-ray-dynamic-image related information on an X-ray dynamic image obtained through X-ray dynamic imaging and camera-moving-image related information on a camera moving image obtained through camera imaging; and
synchronously displaying the X-ray-dynamic-image related information and the camera-moving-image related information.

22. An X-ray-dynamic-image display system comprising:
an imaging apparatus that performs the X-ray dynamic imaging;
a camera that performs the camera imaging; and
the X-ray-dynamic-image display apparatus according to claim 1.
